(19) United States
(12) Patent Application Publication
(10) Pub. No.: US 2004/0153087 A1
(43) Pub. Date: Aug. 5, 2004

(54) Provisional orthopedic implant with removable guide

(76) Inventors: Adam H. Sanford, Warsaw, IN (US); Scott C. Lazar, Cromwell, IN (US); Scott J. Steffensmeier, Warsaw, IN (US); Richard V. Williamson, Ana Cortes, WA (US)

Correspondence Address: BAKER & DANIELS, 111 E. WAYNE STREET, SUITE 800, FORT WAYNE, IN, 46802

(21) Appl. No.: 10/358,010

(22) Filed: Feb. 4, 2003

(51) Int. Cl. A61B 17/90; A61F 2/38; A61F 2/46

(52) U.S. Cl. 606/88; 623/20.3; 606/96

(57) ABSTRACT

A provisional orthopedic prosthetic component and a removable guide element. The provisional component may be mounted to a bone prior to fully preparing the bone for receiving a corresponding permanent prosthetic component to preliminarily assess the fit of the permanent prosthetic component. The guide element is then attached to the provisional component. The guide element is engaged with a surgical tool to position the tool for preparing the bone for the permanent prosthetic component. In an alternative embodiment, the provisional component is an assembly of first and second provisional components. After mounting the provisional assembly on a partially prepared bone, the second provisional component is interchanged with a guide element which is mounted on the first provisional component. The guide element is used to position a surgical tool relative to the first provisional component. Both embodiments may be mounted on a distal femur and define a single-condylar shaped projection.

### BACKGROUND OF THE INVENTION

1. Field of the Invention

The present invention relates to orthopedic prostheses and, more specifically, to provisional components used during orthopedic surgery to facilitate the selection of a permanent orthopedic prosthesis.

2. Description of the Related Art

The use of orthopedic implants to form artificial joints such as prosthetic knees is well known in the art. During a conventional surgical procedure to implant a prosthetic knee joint, a provisional femoral component and a provisional tibial component are placed on the distal femur and proximal tibia after resecting the distal femur and proximal tibia to confirm the proper size and position of the permanent femoral and tibial components. The provisional components typically come in a range of sizes which are identical in size and shape to the permanent components. The provisional components which are placed on the resected femur and tibia are typically selected after making a preliminary determination of the proper size. A trial reduction of the knee joint with the provisional components in place may indicate that the preliminary size determination was incorrect, that the gap between the femur and tibia is insufficient, or some other undesirable characteristic which requires the selection of a different sized tibial or femoral component thereby necessitating the further resection of either the tibia or femur.

Resection of the femur generally involves making three or four intersecting generally planar cuts and making changes to one such cut may require changes in the remaining cuts. The tibial plateau, on the other hand, generally only involves a single cut and provides a more convenient location for corrective actions. It is generally desirable to leave as much healthy bone stock as possible when implanting either total or partial prosthetic knee joints. It is particularly desirable to leave as much healthy bone stock as possible when implanting a unicompartmental or partial knee prosthesis which involves the removal of only a single condylar. This is due to the possibility that the remaining natural condylar may require replacement at a later date and leaving a greater amount of bone stock facilitates the later removal of the partial prosthetic joint and the implantation of a total prosthetic knee joint. Consequently, an improved system which facilitates the proper sizing, selection and positioning of the prosthetic components while minimizing the risk of having to conduct additional bone resections after a trial reduction of the initial provisional components would be desirable.

### SUMMARY OF THE INVENTION

The present invention provides an improved provisional orthopedic prosthesis that may be placed on a partially resected bone to facilitate the proper selection of the permanent prosthesis and thereby minimize the risk of having to make corrective cuts on the partially resected bone. The present invention also provides a removable instrument guide which is securable to the provisional component whereby the provisional component with the attached instrument guide provides a finishing guide engageable with a surgical tool for preparing the bone to receive the permanent prosthetic component.

The invention comprises, in one form thereof, a provisional orthopedic prosthesis for use with a permanent orthopedic prosthesis. The provisional prosthesis includes a provisional component removably mountable on a bone. The provisional component has a first surface facing inwardly relative to the bone and a second surface facing outwardly relative to the bone. The provisional component has a configuration adapted to assess the fit of the permanent orthopedic prosthesis and is moveable through a first range of motion when mounted on the bone. A guide element removably attachable to the provisional component at a predefined position on the provisional component is also provided. The guide element inhibits movement of the provisional component through the first range of motion and is engageable with a surgical tool wherein the guide element positions the surgical tool at a predefined position relative to the provisional component.

The outward facing second surface of the provisional component may define an articulating surface with the guide element being mountable on the second surface. The provisional component may be adapted for mounting on distal femur wherein the articulating surface includes at least one condylar-shaped projection.

The provisional component may also define an opening extending between the first and second surfaces wherein the guide element is mounted proximate the opening and a surgical tool engaged with the guide element extends into the opening in the provisional component. Such an opening in the provisional component may be an elongate opening having a first width wherein the guide element defines a cutting slot positionable over the elongate opening with the cutting slot having a second width which is less than the first width of the elongate opening in the provisional component. The guide element may also include a drill guide opening which defines an axis wherein mounting the guide element on the provisional component positions the drill guide axis so that it extends through the opening in the provisional component.

The guide element and provisional component may also include at least one projection and at least one recess whereby positioning the projection in the recess mounts the guide element to the provisional component. For example, the guide element may include a plurality of projections and the provisional component include a plurality of recesses and positioning the plurality of projections in the plurality of recesses mounts the guide element on the provisional component. Alternative embodiments of the provisional component may have a base portion and a detachable portion wherein the detachable portion and the guide element are interchangeably mountable on the base portion.

The invention comprises, in another form thereof, a provisional orthopedic prosthesis for use with a permanent orthopedic prosthesis. The provisional prosthesis includes a provisional component removably mountable on a bone. The provisional component has a first surface facing inwardly relative to the bone and a second surface that faces outwardly relative to the bone and defines an articulating surface. The provisional component has a configuration adapted to assess the fit of the permanent orthopedic prosthesis and moveable through a first range of motion when mounted on the bone. A guide element removably attachable to the provisional component at a predefined position on the provisional component is also provided. The guide element projects outwardly from the second surface when mounted to the provisional component and is engageable with a surgical tool whereby the surgical tool is positionable at a predefined position relative to the provisional component.

The inward facing first surface may include a first substantially planar portion adapted to engage a resected bone surface and a second non-planar portion adapted to be positioned facing an unresected bone surface. The provisional component may also define a first thickness between the first portion of the inward facing first surface and the outward facing second surface and define a second thickness between the second portion of the inward facing first surface and the outward facing second surface wherein a substantial portion of the first thickness is greater than a substantial portion of the second thickness. Additionally, the guide element may be mountable on the outward facing second surface at least partially opposite the second portion of the inward facing first surface. The provisional component may also define an integral second guide element engageable with a second tool extending between the first portion of the inward facing first surface and the outward facing second surface.

The invention comprises, in yet another form thereof, a provisional orthopedic prosthesis for use with a permanent orthopedic prosthesis. The provisional prosthesis includes a first provisional component and a second provisional component wherein the second provisional component is removably attachable to the first provisional component. The first and second components together define a provisional assembly mountable on a bone. The provisional assembly has a first surface facing inwardly relative to the bone and a second surface facing outwardly relative to the bone and a configuration adapted to assess the fit of the permanent orthopedic prosthesis. A guide element removably attachable to the first provisional component at a predefined position on the first component is also provided. The guide element is engageable with a surgical tool wherein the guide element positions the surgical tool at a predefined position relative to the first provisional component. The guide element and the second provisional component are interchangeably attachable to the first provisional component whereby attachment of one of the second provisional component and the guide element to the first provisional component inhibits attachment of the other of the second provisional component and the guide element to the first provisional component.

The first provisional component may have the first half of a dove tail joint and the second provisional component and guide element each include the second half of a dove tail joint engageable with the first half disposed on the first provisional component. The first provisional component may define a first thickness between the first surface and the second surface and the second provisional component define a second thickness between the first surface and the second surface wherein a substantial portion of the first thickness is greater than a substantial portion of the second thickness, i.e., the first provisional component has a thickness which is generally greater than the thickness of the second provisional component. The first provisional component may also include an integral second guide element engageable with a surgical tool.

The first provisional component may define a first substantially planar portion of the inward facing first surface and the second provisional component define a second substantially non-planar portion of the inner facing first surface. To more firmly secure the guide element to the bone, the guide element may include at least one aperture adapted to receive a securement member such as a holding pin.

The invention comprises, in another form thereof, a method of preparing a bone for a permanent orthopedic prosthesis. The method includes removably mounting a provisional component having an articulating surface to the bone and assessing the fit of the provisional component after mounting the provisional component to the bone including moveably engaging the articulating surface with an opposed surface. The method also includes attaching a guide element to the provisional component wherein the guide element projects outwardly from the articulating surface, engaging a surgical tool with the guide element to thereby position the surgical tool in a predefined location relative to the bone, and preparing the bone to receive the permanent orthopedic prosthesis using the surgical tool.

The method may also include the step of partially resecting the bone prior to mounting the provisional component on the bone. The step of assessing the fit of the provisional component may also include moving the provisional component through a range of motion wherein attaching the guide element to the provisional component inhibits movement of the provisional component through the full range of motion. The provisional component may have an articulating surface defining a single condylar-shaped projection and be mounted on a distal femur.

The invention comprises, in still another form thereof, a method of preparing a bone for a permanent orthopedic prosthesis. The method includes removably mounting to the bone a provisional component assembly having a first provisional component and a second provisional component, each of the first and second provisional components defining a portion of an articulating surface and assessing the fit of the provisional component assembly after mounting the provisional component assembly to the bone including moveably engaging the articulating surface with an opposed surface. The method also includes detaching the second provisional component from the first provisional component and attaching a guide element to the first provisional component, engaging a surgical tool with the guide element to thereby position the surgical tool in a predefined location relative to the first provisional component; and preparing the bone to receive the permanent orthopedic prosthesis using the surgical tool.

The method may also include partially resecting the bone prior to mounting the provisional component assembly on the bone. The step of attaching the guide element to the first provisional component may also include securing the guide element directly to the bone.

An advantage of the present invention is that it provides a provisional component for assessing the fit of a corresponding permanent prosthetic component wherein the provisional component may be mounted on a bone which has only been partially prepared to receive the permanent component and which also provides a removable guide element for engaging a surgical tool. The provisional component and removable guide element thereby act as a finishing guide for preparing the bone to receive the permanent prosthetic component.

### BRIEF DESCRIPTION OF THE DRAWINGS

The above mentioned and other features and objects of this invention, and the manner of attaining them, will become more apparent and the invention itself will be better understood by reference to the following description of the invention taken in conjunction with the accompanying drawings, wherein:

FIG. 1 is a perspective view of a first embodiment of the present invention.

FIG. 2 is a bottom view of the first embodiment.

FIG. 3 is a side view of the first embodiment.

FIG. 4 is a posterior view of the first embodiment.

FIG. 5 is a perspective view of the provisional component of the first embodiment.

FIG. 6 is a side view of the guide element of the first embodiment.

FIG. 7 is a perspective view of a second embodiment of the present invention with the posterior rail component attached thereto.

FIG. 8 is a top view of the second embodiment of FIG. 7.

FIG. 9 is a side view of the second embodiment of FIG. 7.

FIG. 10 is a posterior view of the second embodiment of FIG. 7.

FIG. 11 is a perspective view of the posterior rail of the second embodiment.

FIG. 12 is a perspective view of the second embodiment with a guide element attached thereto.

FIG. 13 is a top view of the second embodiment of FIG. 12.

FIG. 14 is a side view of the second embodiment of FIG. 12.

FIG. 15 is a posterior view of the second embodiment of FIG. 12.

FIG. 16 is a perspective view of the guide element of the second embodiment.

FIG. 17 is a view of a resected tibia and partially resected femur.

FIG. 18 is a side view of a femur and tibia, a permanent orthopedic prosthesis and the outline of a provisional component.

FIG. 19 is a side view of the first embodiment attached to a femur.

FIG. 20 is a side view of a femur and a femoral component of a permanent orthopedic prosthesis.

Corresponding reference characters indicate corresponding parts throughout the several views. Although the exemplification set out herein illustrates the invention in several forms, the embodiments disclosed below are not intended to be exhaustive or to be construed as limiting the scope of the invention to the precise forms disclosed.

### DESCRIPTION OF THE PRESENT INVENTION

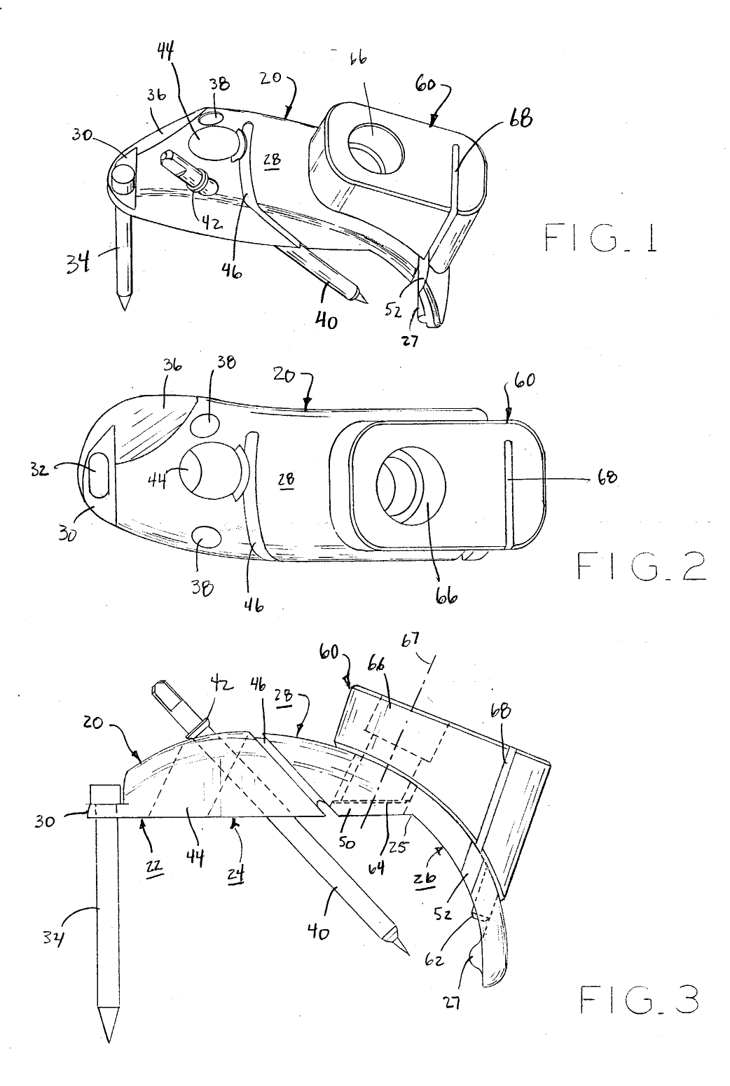

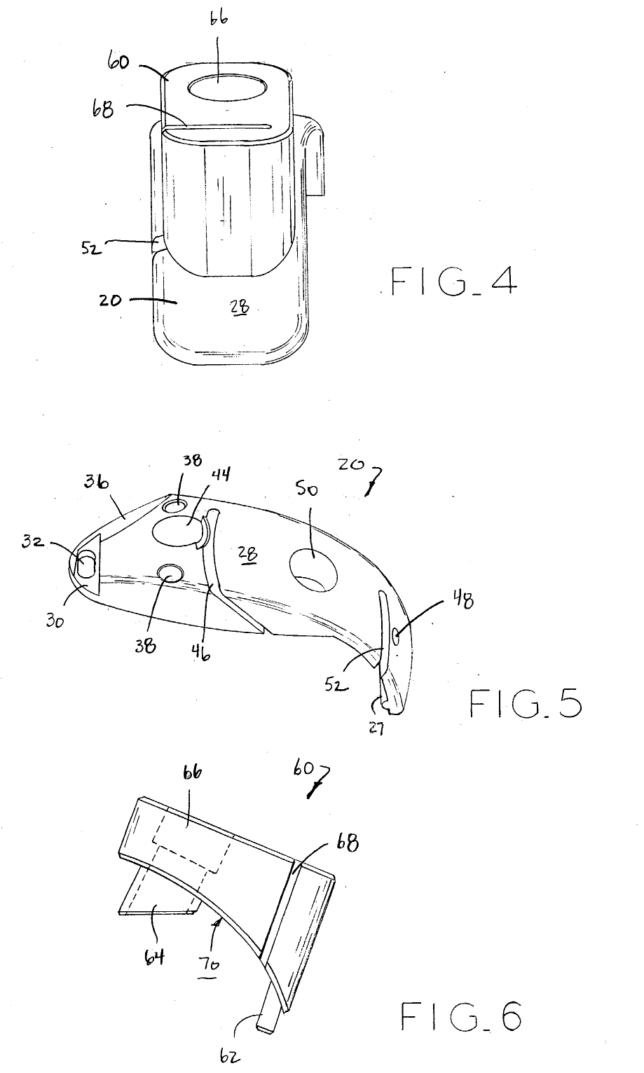

A stainless steel provisional component 20 and guide element 60 in accordance with present invention are illustrated in FIG. 1. Provisional component 20 is mountable on a distal femur and has a first surface 22 which faces inwardly relative to distal femur when mounted thereon. As seen in FIG. 3, first surface 22 has a first substantially planar portion 24 and a second non-planar portion 26 which meet at intersection 25. A small projecting rib 27 is located on second portion 26 of inner surface 22 near the posterior end of provisional component 20. Provisional component 20 also includes a second surface 28 which faces outwardly relative to the distal femur on which provisional component 20 is mounted. The illustrated provisional component 20 is adapted for use in a partial knee prosthesis and surface 28 defines an articulating surface having a single condylar-shaped projection.

The anterior end of provisional component 20 includes a projecting lip 30 which has an elongated opening 32. As discussed in greater detail below, a bone screw 34 positioned within opening 32 is used to secure provisional component 20 to femur 72. Bone screw 34 has a conventional construction and is only schematically represented in FIGS. 1 and 3. Located adjacent lip 30 is a cut-out portion 36 which provides clearance for the patella when provisional component 20 is mounted on a distal femur. Provisional component also includes a pair of openings 38 which extend from outer surface 28 to the first planar portion 24 of inner surface 22. As can be seen in FIGS. 1 and 3, a conventional holding pin 40 with a removable collar 42 is insertable through openings 38. An integral drill guide 44 is located between openings 38. An integral cutting guide 46 is located proximate drill guide 44. Both drill guide 44 and cutting guide 46 are located in a relatively thick section of provisional component 20 and define openings which extend from outer surface 28 to first portion 24 of inner surface 22.

Mounting recesses 48 and 50 are shown in FIG. 5. An elongate opening 52 is located between recesses 48 and 50. As best seen in FIG. 6, guide element 60 includes two projections 62 and 64 which are inserted into recesses 48 and 50 respectively to mount guide element 60 to second surface 28. An opening having two cylindrical sections with differing diameters forms drill guide 66 which extends co-axially through cylindrical projection 64. Drill guide opening 66 defines an axis 67 which extends co-axially through opening 50 in provisional component 20 when guide element 60 is mounted to provisional component 20. Guide element 60 also includes a cut slot 68 which is positioned over elongate opening 52 when guide element 60 is mounted to provisional component 20. Lower surface 70 of guide element 60 generally conforms to outer surface 28 of provisional component 20 and is engaged therewith when guide element 60 is mounted to provisional component 20.

Provisional component 20 defines a first thickness between first portion 24 of inner surface 22 and outer surface 28 which is generally greater than a second thickness defined by provisional component 20 between second portion 24 of inner surface 22 and outer surface 28. The greater first thickness allows drill guide 44 and cut slot 46 to be formed integrally in provisional component 20 between first inner surface portion 24 and outer surface 28. The relatively thinner thickness defined by provisional component 20 between second inner surface portion 26 and outer surface 28 and between first inner surface portion 24 and outer surface 28 proximate intersection 25 makes this section of provisional component 20 less suitable for providing an integral stabilizing guide for a surgical drill or cutting blade. If a drill or cutting blade were inserted into a guide hole or guide slot of a relatively shallow depth, the drill or cutting blade would be more likely to tilt relative to the guide hole or slot. Guide element 60 increases the effective thickness of provisional component 20 and thereby provides more effective guide elements such as drill guide 66 and cutting slot 68.

The use of provisional component 20 and guide element 60 will now be described with reference to FIGS. 17-20. FIG. 17 illustrates a right femur 72 and tibia 56 in flexion after performing a resecting the right lateral condyle on femur 72 thereby forming distal cut surface 74 and resecting the right lateral portion of the tibial plateau leaving surface 56. After partially resecting femur 72 and tibia 56 as shown in FIG. 17, provisional component 20 is mounted on femur 72.

Provisional component 20 mounted on femur 72 in the position shown in dashed outline in FIG. 18. FIG. 18 also illustrates permanent femoral component 80 in its implanted position on femur 72. One or more bone screws 34 or holding pins 40 are inserted through opening 30 to secure provisional component 20 to femur 72 after locating provisional component 20 in its desired position. By providing an elongated opening 30 which extends in the lateral/medial direction, opening 30 allows the position of provisional component 20 to be conveniently adjusted by translation in the lateral/medial direction as well as rotation about the shaft of bone screw 34 after partially attaching bone screw 34 to femur and prior to tightly securing provisional component 20 to femur 72 with bone screw 34.

After provisional component 20 is secured to femur 72 with bone screw 34, a trial reduction is performed. In other words, femur 72 and tibia 46 are re-engaged and moved through a range of motion from a flexion position to an extension position to assess the fit of provisional component 20. When femur 72 and tibia 54 are in flexion the anatomical axis of femur 72 defined by the femoral medullary canal is aligned with line 72f and femur 72 and tibia 54 are positioned as shown in FIGS. 17 and 18. When femur 72 and tibia 54 are in extension, the anatomical axis of femur 72 is in the position indicated by line 72e.

Provisional component 20 is mounted on femur 72 after only one of three resections has been performed on femur 72. To facilitate the mounting of provisional component 20 on a partially resected femur, second portion 26 of inner surface 22 is spaced outwardly to provide clearance for femur 23. Non-planar second inner surface portion 26 is also configured to generally track the outer surface of femur 72 with rib 27 being adapted to maintain second inner surface portion 26 at a desired spacing from the outer surface of the unresected posterior of femur 72.

Substantially planar first inner surface portion 24 is adapted to engage the planar surface of distal cut 74. The outer articulating surface 81 of permanent femoral component 80 is configured to generally replicate the original outer surface of femur 72. As best seen in FIG. 18, outward facing articulating surface 28 of provisional component 20 projects a greater distance outwardly from femur 72 than articulating surface 81. This differs from typical provisional components which are substantially similar in size and shape as their corresponding permanent components.

Despite its larger outer surface, provisional component 20 can still be used to assess the fit of permanent femoral component 80. By configuring outer surface 28 such that it projects outwardly by a substantially constant value toward tibia 54 and using a provisional tibial component 90 which has a thickness which is less than the thickness of permanent tibial component 82 by that same value, provisional component 20 can be used to assess whether preliminarily selected permanent components 80, 82 are the proper selection. FIG. 18 schematically illustrates tibial provisional component 90 in dashed outline. Provisional component 90 includes a lower surface 94 engageable with tibial surface 56 and an upper surface 92 bearingly engageable with provisional component 20. In the illustrated embodiment, the distance by which surface 28 projects outwardly relative to surface 81 may advantageously fall within the range of 6 to 8 mm. In an alternative embodiment, tibial provisional component 90 may be entirely omitted by having outer surface 28 project a distance outwardly relative to surface 81 which is substantially equivalent to the thickness of permanent tibial component 82.

During the trial reduction of provisional component 20, the surgeon will move femur 72 and tibia 54 between flexion and extension and examine the various attributes of the joint including tightness of the ligaments and other soft tissues in the same manner that would be employed if conventional provisional components having the same shape and size as the permanent components had been mounted to femur 72 and tibia 54.

Outer surface 81 of femoral component 80 defines a condyle-shaped projection forming an articulating surface that bears against an opposed bearing surface 85 on tibial component 82. In actual use, the motion of femoral component 80 relative to tibial component 82 is primarily rotational but also includes a relatively small anterior/posterior sliding component. Because of the complex nature of outer surface 81 and of the relative rotational and sliding motion of femoral component 80, provisional component 20 with its larger articulating surface 28 provides only an approximation of the fit that will be realized by the implantation of femoral component 80 and tibial component 82. This approximation, however, is considered sufficiently accurate to provide a meaningful assessment of the likely fit of femoral component 80 and tibial component 82. The use of a provisional orthopedic prosthetic component to assess the fit of a smaller permanent prosthetic component is described by Sanford et al. in a U.S. Patent Application entitled PROVISIONAL ORTHOPEDIC PROSTHESIS FOR PARTIALLY RESECTED BONE having an attorney docket number of ZIM0152 and filed on the same date as the present application and is hereby expressly incorporated herein by reference.

If the assessment of provisional component 20 is positive, guide element 60 is mounted on provisional component 20 and component 20 is more firmly secured to femur 72 by inserting two holding pins 40 into femur 72 through openings 38. Guide element 60 projects outwardly from articulating surface 28 when mounted on provisional component 20, thus, femur 72 must be placed in a position, e.g., flexion, which allows for the mounting of guide element 60 to provisional component 20. After mounting guide element 60, drill bit 102 of drill 100 is inserted into and engaged with drill guides 44 and 66 to properly position drill 100 relative to provisional component 20 when forming bores 43, 65 in femur 72. Similarly, cutting blade 98 of cutting instrument 96 is inserted into and engaged with cutting slots 46 and 68 to properly position cutting instrument 96 when forming chamfer cut 76 and posterior cut 78 respectively. Due to the presence of holding pins and bone screws, it may be necessary to complete chamfer cut 76 and posterior cut 78 after removing provisional component 20 and guide element 60 from femur 72.

After removing all bone screws and holding pins and provisional component 20 and guide element 60 from femur 72, any extraneous bone located on distal cut 74, chamfer cut 76, and posterior cut 78 is removed to prepare femur 72 to receive permanent implant 80. A final provisional component having a configuration and size which is identical to permanent component 80 is then mounted on femur 72 by inserting mounting posts located on the provisional component into bores 43, 65 on femur 72. A provisional tibial component having the same thickness as permanent component 82 is placed on surface 56 and the fit of components 80, 82 is assessed again prior to implanting components 80, 82. A final provisional component which provides for the mounting of a guide instrument to femur 72 for recutting femur 72 in the event that the provisional component indicates that a different sized femoral component is required is described by Sanford in a U.S. patent application entitled PROVISIONAL ORTHOPEDIC IMPLANT AND RECUTTING INSTRUMENT GUIDE having attorney docket number ZIM0154 and filed on the same date as the present application and which is hereby expressly incorporated herein by reference.

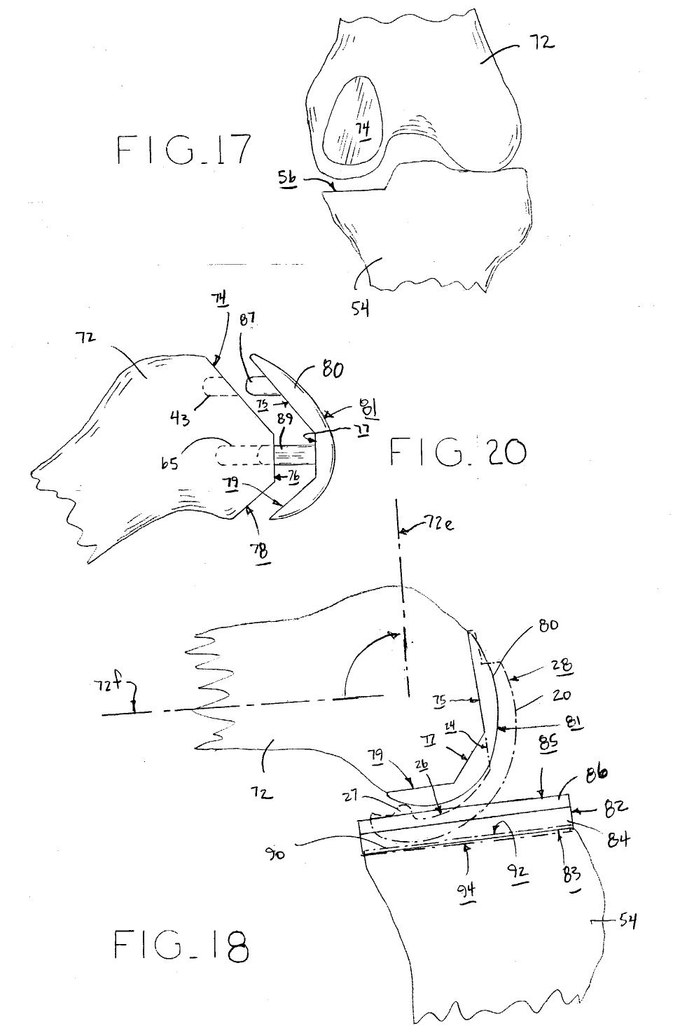

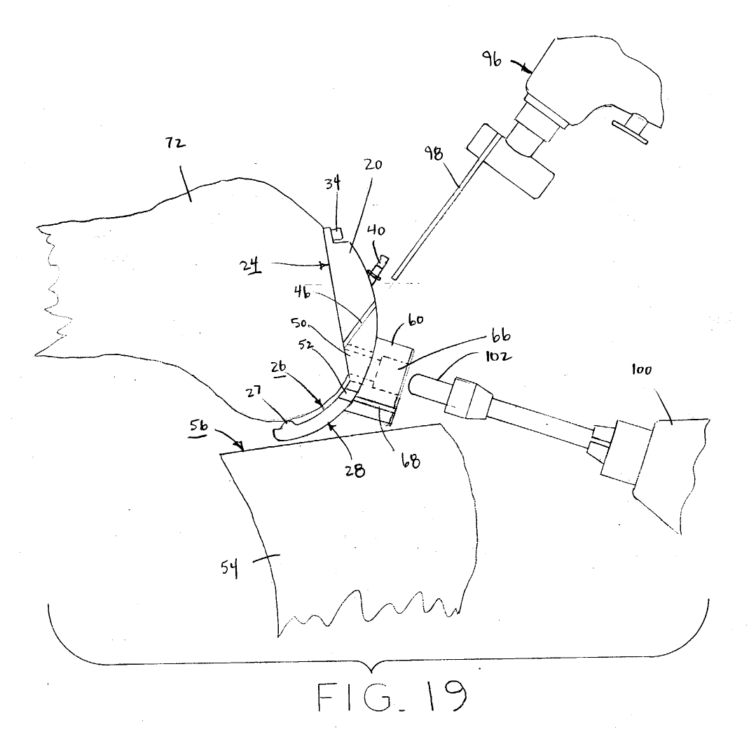

If the trial reduction using the final provisional components is successful, the final provisional components are removed and tibial component 82 and femoral component 80 are implanted on tibia 54 and femur 72 respectively. As can be seen in FIG. 20, femoral component 80 includes inward facing surfaces 75, 77 and 79 which respectively engage distal cut 74, chamfer cut 76 and posterior cut 78. Femoral component 80 also includes mounting posts 87, 89 which are inserted into bores 43, 65 respectively. Tibial component 82 includes a metallic base component 84 having a lower surface 83 mounted on tibial surface 56 and a polyethylene bearing member 86 which defines bearing surface 85 engageable with femoral component 80. Tibial component 82 and femoral component 80 are implanted in a conventional manner employing bone cement. In the illustrated embodiment, prosthetic components 54, 72 are commercially available components sold under the name M/G™ Unicompartmental Knee by Zimmer Inc. of Warsaw, Ind.

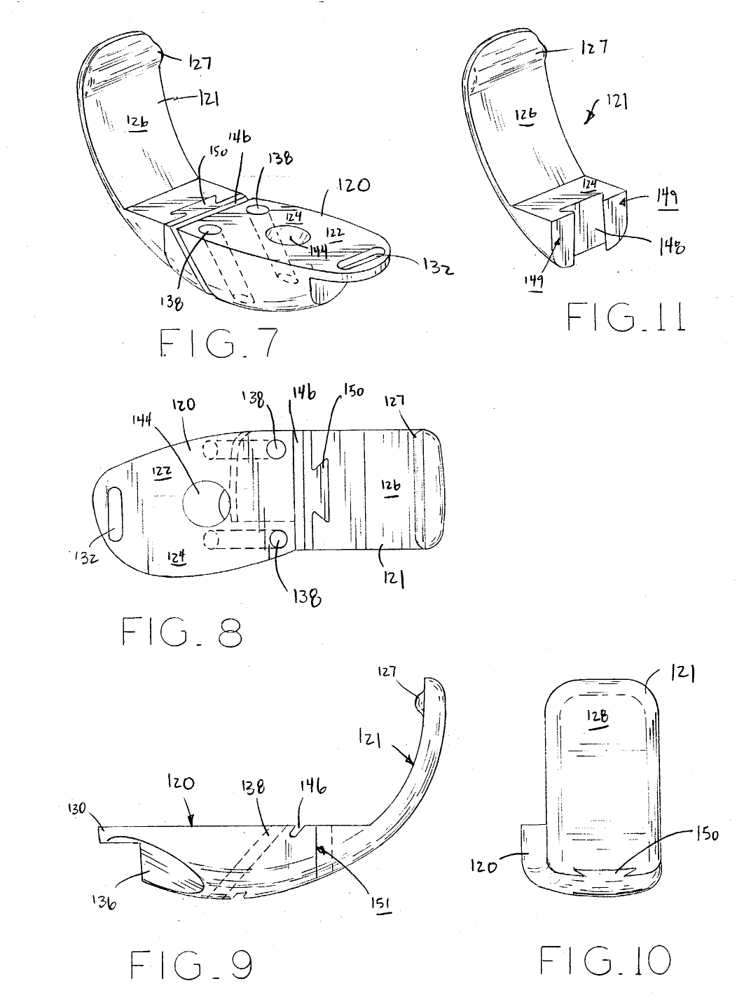

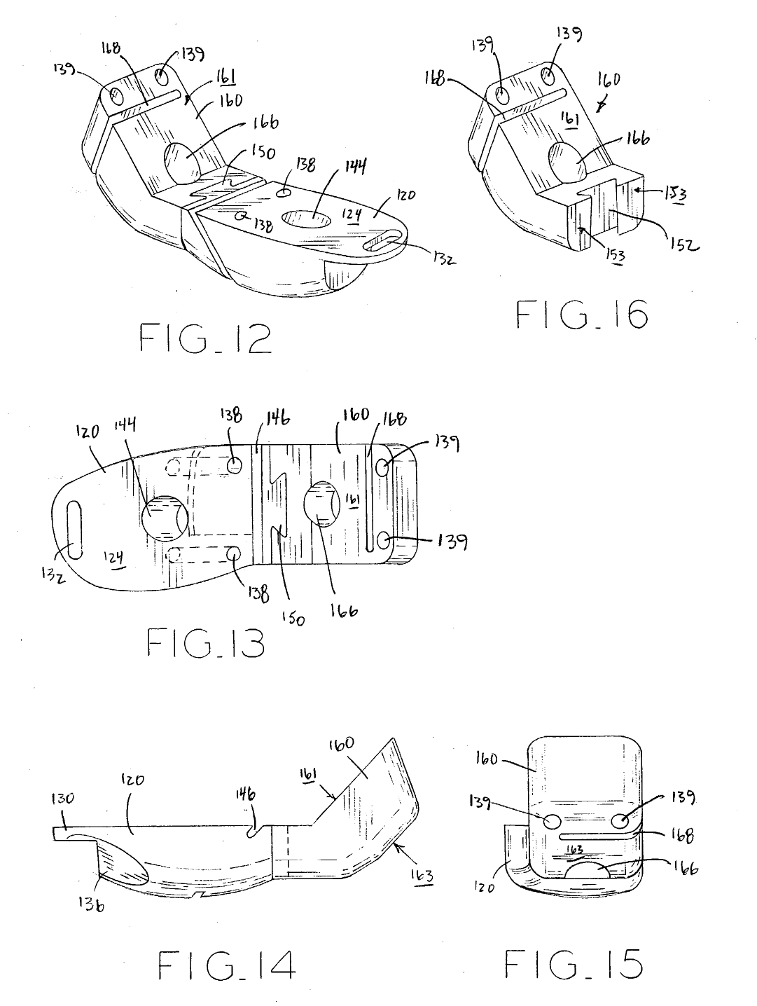

A second embodiment of the present invention is illustrated in FIGS. 7-16. The second embodiment includes a first provisional component 120 and a second provisional component 121. The second or detachable component 121 is detachably secured to the first or base provisional component 120 with a dovetail joint. When attached together, first and second provisional components 120, 121 form a provisional component assembly which functions in the same manner as provisional component 20 without guide element 60 attached thereto. More specifically, the provisional assembly formed by provisional components 120, 121 and illustrated in FIGS. 7-10 includes a inward facing surface 122 having a substantially planar first portion 124 and a non-planar second portion 126 and an oppositely disposed outward facing surface 128. Outer surface 128 defined by first and second components 120, 121 forms an articulating surface defining a single condylar-shaped projection similar to surface 28 of provisional component 20. Other features located on provisional assembly 120, 121 that are common to both provisional assembly 120, 121 and provisional component 20 are rib 127, projecting anterior lip 130, elongate opening 132, openings 138, integral drill guide 144 and integral cut slot 146. These features are discussed above with reference to provisional component 20.

As seen in FIGS. 7-11, a male dove tail joint 150 is located on first provisional component 120 and a female dove tail joint 148 is located on second provisional component 121. Surfaces 151 are located adjacent male joint 150 on first provisional component 120 and engage surfaces 149 adjacent female joint 148 when joints 148 and 150 are engaged to mount second provisional component 121 on first provisional component 121. Guide element 160 also includes a female dove tail joint 152 with surfaces 153 disposed adjacent thereto which have the same configuration as female joint 148 and surfaces 149 whereby second provisional component 121 and guide element 160 are interchangeably mountable on first provisional component 120. Dove tail joints 150/ 148, 152 or engagement surfaces 151/149, 153 may include a taper to facilitate the proper positioning of second provisional component 121 and guide element 160 on first provisional component 120. As can be seen in FIGS. 7-11, first portion 124 of inner surface 122 is primarily located on first provisional component 120 and second provisional component 121 defines only a small portion of first portion 124 and all of second portion 126 of inner surface 122. First and second inner surface portions 124, 126 are similar to inner surface portions 24, 26 defined by provisional component 20. Consequently, first provisional component 120 defines a thickness between inner surface 122 and outer surface 128 which is generally greater than that defined by second provisional component 121. The increased thickness of first component 120 allows first provisional component 120 to define integral drill guide 144 and integral cut slot 146.

Guide element 160 is illustrated in FIGS. 12-16 and includes a drill guide 166 and cutting slot 168 which extend between inward facing surface 161 and outward facing surface 163 and which are engageable with a cutting drill 100 and cutting instrument 96 respectively. Surfaces 161 and 163 define a thickness therebetween which is sufficient to allow drill guide 166 and cutting slot 168 to effectively engage and position drill 100 and cutting instrument 96. Guide features 166, 168 position these surgical tools in a predefined position relative to provisional component 120 and guide element 160 thereby acts as a finishing guide when preparing femur 72 to receive permanent femoral component 80. Apertures 139 are also provided on guide element 160 for receiving holding pins 40. First and second provisional components 120, 121 and guide element 160 may all be manufactured of stainless steel materials.

The use of provisional components 120, 121 and guide element 160 will now be discussed. Provisional assembly 120,121 is mounted on a partially resected femur and used to assess the fit of permanent femoral component 80 in the same manner as provisional component 20. If the assessment is positive, holding pins 40 are secured to femur 72 through openings 138 and second provisional component 121 is detached from first provisional component 120. Guide element 160 is then mounted on first provisional component 120. Guide element 160 may also be directly secured to femur 72 by inserting holding pins 40 into femur 72 through openings 139. Femur 72 is then prepared to receive permanent femoral component 80 using drill guides 144 and 166 to position drill 100 and cut slots 146 and 168 to position cutting instrument 96. The bone screws and holding pins attaching first provisional component 120 and guide element 160 are then removed from femur 72 together with first provisional component 120 and guide element 160. Any extraneous bone material left on femur 72 is then removed and a final provisional component is mounted on femur 72 and tibia 54 before implanting components 80, 82 as discussed above with reference to the first embodiment.

Although the two illustrated embodiments are both configured for use with a partial knee orthopedic prosthesis, the present invention may also be adapted for use with total knee prostheses and orthopedic prostheses implanted at other anatomical locations.

While this invention has been described as having an exemplary design, the present invention may be further modified within the spirit and scope of this disclosure. This application is therefore intended to cover any variations, uses, or adaptations of the invention using its general principles.

What is claimed is:

1. A provisional orthopedic prosthesis for use with a permanent orthopedic prosthesis, said provisional prosthesis comprising:
a provisional component removably mountable on a bone, said provisional component having a first surface facing inwardly relative to the bone and a second surface facing outwardly relative to the bone, said provisional component having a configuration adapted to assess the fit of the permanent orthopedic prosthesis and moveable through a first range of motion when mounted on the bone; and
a guide element removably attachable to said provisional component at a predefined position on said provisional component, said guide element inhibiting movement of said provisional component through said first range of motion and engageable with a surgical tool wherein said guide element positions the surgical tool at a predefined position relative to said provisional component.

2. The provisional orthopedic prosthesis of claim 1 wherein said second surface defines an articulating surface and said guide element is mountable on said second surface.

3. The provisional orthopedic prosthesis of claim 1 wherein said provisional component defines an opening extending between said first and second surfaces and said guide element is mountable proximate said opening and wherein a surgical tool engaged with said guide element is positionable within said opening.

4. The provisional orthopedic prosthesis of claim 3 wherein said opening in said provisional component is an elongate opening having a first width and said guide element defines a cutting slot positionable over said elongate opening, said cutting slot having a second width which is less than said first width.

5. The provisional orthopedic prosthesis of claim 3 wherein said guide element includes a drill guide opening defining an axis and mounting of said guide element on said provisional component positions said drill guide opening wherein said axis extends through said opening in said provisional component.

6. The provisional orthopedic prosthesis of claim 1 wherein said provisional component is adapted for mounting on a distal femur and said second surface defines an articulating surface including at least one condylar-shaped projection, said guide element mountable on said second surface.

7. The provisional orthopedic prosthesis of claim 1 wherein said guide element and said provisional component further include at least one projection and at least one recess whereby positioning said projection in said recess mounts said guide element to said provisional component.

8. The provisional orthopedic prosthesis of claim 1 wherein said guide element includes a plurality of projections and said provisional component includes a plurality of recesses wherein positioning said plurality of projections in said plurality of recesses mounts said guide element on said provisional component.

9. The provisional orthopedic prosthesis of claim 1 wherein said provisional component includes a base portion and a detachable portion, said detachable portion and said guide element being interchangeably mountable on said base portion.

10. A provisional orthopedic prosthesis for use with a permanent orthopedic prosthesis, said provisional prosthesis comprising:
a provisional component removably mountable on a bone, said provisional component having a first surface facing inwardly relative to the bone and a second surface facing outwardly relative to the bone, said second surface defining an articulating surface, said provisional component having a configuration adapted to assess the fit of the permanent orthopedic prosthesis and moveable through a first range of motion when mounted on the bone; and
a guide element removably attachable to said provisional component at a predefined position on said provisional component, said guide element projecting outwardly from said second surface when mounted to said provisional component and wherein said guide element is engageable with a surgical tool whereby the surgical tool is positionable at a predefined position relative to said provisional component.

11. The provisional orthopedic prosthesis of claim 10 wherein said guide element further comprises at least one projection and said provisional component defines at least one recess wherein positioning said at least one projection in said at least one recess mounts said guide element to said provisional component.

12. The provisional orthopedic prosthesis of claim 10 wherein said provisional component defines an opening extending between said first and second surfaces, said guide element being mountable proximate said opening wherein a surgical tool engaged with said guide element is positionable within said opening.

13. The provisional orthopedic prosthesis of claim 10 wherein said first surface includes a first substantially planar portion adapted to engage a resected bone surface and a second non-planar portion adapted to be positioned facing an unresected bone surface.

14. The provisional orthopedic prosthesis of claim 13 wherein said provisional component defines a first thickness between said first portion of said first surface and said second surface and said provisional component defines a second thickness between said second portion of said first surface and said second surface, a substantial portion of said first thickness being greater than a substantial portion of said second thickness, said guide element being mountable on said second surface at least partially opposite said second portion of said first surface.

15. The provisional orthopedic prosthesis of claim 14 wherein said provisional component defines an integral second guide element engageable with a surgical tool, said integral second guide element extending between said first portion of said first surface and said second surface.

16. A provisional orthopedic prosthesis for use with a permanent orthopedic prosthesis, said provisional prosthesis comprising:
a first provisional component and a second provisional component, said second provisional component removably attachable to said first provisional component, said first and second components together defining a provisional assembly mountable on a bone, said provisional assembly having a first surface facing inwardly relative to the bone and a second surface facing outwardly relative to the bone, said provisional assembly having a configuration adapted to assess the fit of the permanent orthopedic prosthesis; and
a guide element removably attachable to said first provisional component at a predefined position on said first component, said guide element being engageable with a surgical tool wherein said guide element positions the surgical tool at a predefined position relative to the first provisional component, said guide element and said second provisional component being interchangeably attachable to said first provisional component whereby attachment of one of said second provisional component and said guide element to said first provisional component inhibits attachment of the other of said second provisional component and said guide element to said first provisional component.

17. The provisional orthopedic prosthesis of claim 16 wherein said first provisional component includes a first half of a dove tail joint and said second provisional component and said guide element each include a second half of a dove tail joint engageable with said first half disposed on said first provisional component.

18. The provisional orthopedic prosthesis of claim 16 wherein said first provisional component defines a first thickness between said first surface and said second surface and said second provisional component defines a second thickness between said first surface and said second surface, a substantial portion of said first thickness being greater than a substantial portion of said second thickness.

19. The provisional orthopedic prosthesis of claim 18 wherein said first provisional component includes an integral second guide element engageable with a surgical tool.

20. The provisional orthopedic prosthesis of claim 16 wherein said first provisional component defines a first substantially planar portion of said first surface and said second provisional component defines a second substantially non-planar portion of said first surface.

21. The provisional orthopedic prosthesis of claim 16 wherein said guide element further comprises at least one aperture adapted to receive a securement member.

22. A method of preparing a bone for a permanent orthopedic prosthesis, said method comprising:
removably mounting a provisional component having an articulating surface to the bone;
assessing the fit of the provisional component after mounting the provisional component to the bone including moveably engaging the articulating surface with an opposed surface;
attaching a guide element to the provisional component wherein the guide element projects outwardly from the articulating surface;
engaging a surgical tool with the guide element to thereby position the surgical tool in a predefined location relative to the bone; and
preparing the bone to receive the permanent orthopedic prosthesis using the surgical tool.

23. The method of claim 22 further comprising the step of partially resecting the bone prior to mounting the provisional component on the bone.

24. The method of claim 22 wherein said step of assessing the fit of the provisional component includes moving the provisional component through a range of motion and wherein said step of attaching the guide element to the provisional component inhibits movement of the provisional component through the range of motion.

25. The method of claim 22 wherein the articulating surface of the provisional component defines a single condylar-shaped projection and said step of removably mounting the provisional component to the bone includes mounting the provisional component to a distal femur.

26. A method of preparing a bone for a permanent orthopedic prosthesis, said method comprising:
removably mounting to the bone a provisional component assembly having a first provisional component and a second provisional component, each of the first and second provisional components defining a portion of an articulating surface;
assessing the fit of the provisional component assembly after mounting the provisional component assembly to the bone including moveably engaging the articulating surface with an opposed surface;
detaching the second provisional component from the first provisional component and attaching a guide element to the first provisional component;
engaging a surgical tool with the guide element to thereby position the surgical tool in a predefined location relative to the first provisional component; and
preparing the bone to receive the permanent orthopedic prosthesis using the surgical tool.

27. The method of claim 26 further comprising the step of partially resecting the bone prior to mounting the provisional component assembly on the bone.

28. The method of claim 26 wherein attaching the guide element to the first provisional component further comprises securing the guide element directly to the bone.

29. The method of claim 26 wherein the articulating surface of the provisional component assembly defines a single condylar-shaped projection and said step of removably mounting the provisional component to the bone includes mounting the provisional component assembly to a distal femur.
